(19) United States
(12) Patent Application Publication
(10) Pub. No.: US 2010/0225476 A1
(43) Pub. Date: Sep. 9, 2010

(54) PATCH FOR DETECTING MOVEMENTS OF A BODY

(76) Inventor: Hans-Peter KLOSE, Stuttgart (DE)

Correspondence Address: KENYON & KENYON LLP, ONE BROADWAY, NEW YORK, NY, 10004, US

(21) Appl. No.: 12/652,888

(22) Filed: Jan. 6, 2010

(30) Foreign Application Priority Data
Mar. 9, 2009 (DE) 102009001398.9

(51) Int. Cl. G08B 21/00; G01P 15/18

(52) U.S. Cl. 340/540; 73/510

(57) ABSTRACT

A patch for detecting movements of a body and its use.

### BACKGROUND INFORMATION

Devices for detecting movements are used in different areas, e.g., in sports, the fitness industry, and in medicine. These products aim mostly at movements during running, movements during walking and similar movements.

German Patent No. DE 101 64 534 describes, for example, a device for determining movement characteristics of a human body or an animal body, which includes a sensor system fastenable to the body, an analyzing device separate from the sensor system, and transmitting means for transmitting signals to the analyzing device. However, the device described in German Patent No. DE 101 64 534 is difficult to handle, clean, and maintain, in particular due to the plurality of individual elements.

### SUMMARY OF THE INVENTION

The subject matter of the present invention is a patch for detecting movements of a body, in particular a human or animal body, e.g., a patient, which has a motion sensor, an analyzing device, a data transmitting device, and a power supply device.

The patch according to the present invention has the advantage that multiple functions are combined in one element. In this way, individual function elements cannot get lost and handling is simplified.

Integrating an analyzer device into the patch has the additional advantage that compatibility problems with connected devices are reduced or even avoided in the event of changes to or refinements of the patch or the analyzing method. Among other things, a plurality of differently specialized patches according to the present invention may be read out using a simple query device, e.g., for sports, rehabilitation, medication adjustment, coma monitoring and tremor measurement.

The design as a patch enables easy attachment, even with loosely fitting clothing (hospital clothing), in particular underneath the clothing, in the event of restricted movements, in particular impaired fine motoricity of the person applying the patch/patient and in the event of mechanical stresses due to movements when lying down. In addition, no help by the patient is necessary in attaching the patch. Moreover, a displacement or change in position of the motion sensor may be avoided by using a patch. Furthermore, pressure points on the patient's body are reduced or even avoided and the wearing comfort is improved. In particular, the patient's sleep is not affected or is barely affected. Furthermore, repeated attaching/removing during the course of the day may be avoided.

The patch according to the present invention may basically be attached to any part of the body, e.g., the abdomen, the arms, or the legs.

Within the scope of a preferred specific embodiment of the present invention, the patch is a disposable patch. This is particularly advantageous, especially for sanitary reasons. After use, the patch is pulled off and discarded, thereby omitting cleaning, charging and/or maintenance. This yields the advantage that the burden on nursing staff may be reduced.

The motion sensor is preferably designed for measuring movements of a body and for outputting measured data, in particular to the analyzer device or a data storage unit. In particular, the motion sensor may be an acceleration sensor. The motion sensor, in particular the acceleration sensor, is preferably a micro-electromechanical system (MEMS). “Micro-electromechanical systems” are understood to be micro-electromechanical systems (MEMS) and also smaller electromechanical systems such as nano-electromechanical systems (NEMS). Furthermore, the motion sensor, in particular the acceleration sensor, is designed for detecting accelerations in two, in particular three, dimensions.

The analyzer device is preferably designed for receiving and processing measured data from the motion sensor. In addition, the analyzer device is preferably designed for outputting analyzed data to the data transmitting device. The measured data of the motion sensor may be processed by the analyzer device with respect to different criteria. Criteria may include for example: movement patterns (e.g., walking, running, lying down), occurring or non-occurring movement (e.g., of sleeping, bed-ridden, dementia, or coma patients) and/or length of movement phases. The analyzer device may include an integrated circuit (IC), e.g., an application-specific integrated circuit (ASIC), in particular a microprocessor. In particular, the analyzer device may be an integrated circuit (IC), e.g., an application-specific integrated circuit (ASIC), in particular a microprocessor.

Within the scope of one specific embodiment, the patch furthermore includes a data storage unit. The data storage unit may be integrated into the analyzer device. The data storage unit is preferably designed for storing, in particular buffering, measured data from the motion sensor and/or processed data from the analyzer device. In this way, data may advantageously be collected over a longer period of time and the number of queries may be reduced.

The data transmission device is preferably designed for receiving processed data from the analyzer device and for, in particular, wireless transmission of processed data to an external query device, for example. The data transmission device may include an antenna or may be designed as an antenna. In particular, the data transmission device may be a transponder, in particular an RFID transponder. The term RFID means: Identification with the aid of electromagnetic waves (Radio Frequency Identification, RFID). By configuring the data transmission device as a transponder, in particular an RFID transponder, the data transmission device may also use already present elements, e.g., an integrated circuit of the analyzer device and/or a data storage unit.

The data may be read out preferably by a stationary query device, e.g., a base station or by a mobile, e.g., handheld reader or a cell phone. The query device may have a display device and/or an acoustic alarm device and/or a visual alarm device. In particular, the query device may be an RFID reader.

The power supply device is preferably designed for the direct or indirect power supply of the motion sensor, the analyzer device, and the data transmission device. Direct power supply is understood in particular that the motion sensor, the analyzer device, and the data transmission device are each supplied with power from the power supply device via its own electrical line. Indirect power supply is understood in particular that at least one of these components, e.g., the motion sensor or the data transmission device, is supplied with power from the power supply device via another component, e.g., the analyzer device. The power supply device may be a battery. A battery is particularly advantageous for use with essentially resting bodies. However, the power supply device may also be a mechanical-electrical energy converter, in particular a piezoelectric generator. This is particularly advantageous for use on intensively moving bodies, e.g., in sports.

Within the scope of another preferred specific embodiment of the present invention, the motion sensor and the data transmission device and the power supply device and the analyzer device and possibly the data storage unit are designed to be bendable. In particular, the motion sensor and the data transmission device and the power supply device and the analyzer device and possibly the data storage unit are designed as a bendable, e.g., one-piece, electronic module. This may be implemented, among other things, by a shared bendable or flexible printed circuit board.

The patch has preferably an adhesive layer. The adhesive layer is preferably a skin-tolerant, preferably allergen-free adhesive agent.

Moreover, the patch may have a supporting layer. In particular, the patch may have a supporting layer which is provided with an adhesive layer. The supporting layer may be made from a textile fabric, for example. The motion sensor and the data transmission device and the power supply device and the analyzer device and possibly the data storage unit may be situated on the supporting layer, in particular opposite the adhesive layer.

Within the scope of a further preferred specific embodiment of the present invention, the patch has a sealing layer. The sealing layer makes it possible to advantageously protect the motion sensor and the data transmission device and the power supply device and the analyzer device and possibly the data storage unit or the electronic module from external, e.g., mechanical or chemical, effects, and the wearing comfort for the patient is improved. The sealing layer is preferably bendable and/or flexible and/or elastic. The motion sensor and the data transmission device and the power supply device and the analyzer device and possibly the data storage unit may be situated in the sealing layer or may be covered by the sealing layer. The electronic module in particular may be situated in the sealing layer or may be covered by the sealing layer. In particular, the patch may have a sandwich-like construction in which the adhesive layer, the supporting layer, and the sealing layer rest on one another in a sandwich-like manner. The supporting layer may be situated between the sealing layer and the adhesive layer, for example. However, it is also possible within the scope of the present invention to omit the supporting layer. For example, the patch may have a sealing layer which is provided with an adhesive layer or which has an adhesive layer.

Within the scope of a further specific embodiment of the present invention, the motion sensor and the data transmission device and the power supply device and the analyzer device and possibly the data storage unit are integrated into a housing.

Another subject matter of the present invention is the use of a patch according to the present invention
as a disposable patch and/or
for detecting movements of a human or an animal, in particular humans, e.g., in the area of sports, the fitness industry, in elderly care, or in medicine, and/or
for monitoring movements of sleeping, bed-ridden, dementia, or coma patients, and/or
for minimizing the spontaneous movement, and/or
for measuring muscular tremors, and/or
for medication adjustment of Parkinson's patients.

### BRIEF DESCRIPTION OF THE DRAWINGS

FIG. 1 shows a schematic cross section through an electronic module of a patch according to the present invention.

FIG. 2 shows a schematic, perspective view of a first specific embodiment of a patch according to the present invention.

FIG. 3 shows a schematic cross section through a second specific embodiment of a patch according to the present invention.

### DETAILED DESCRIPTION

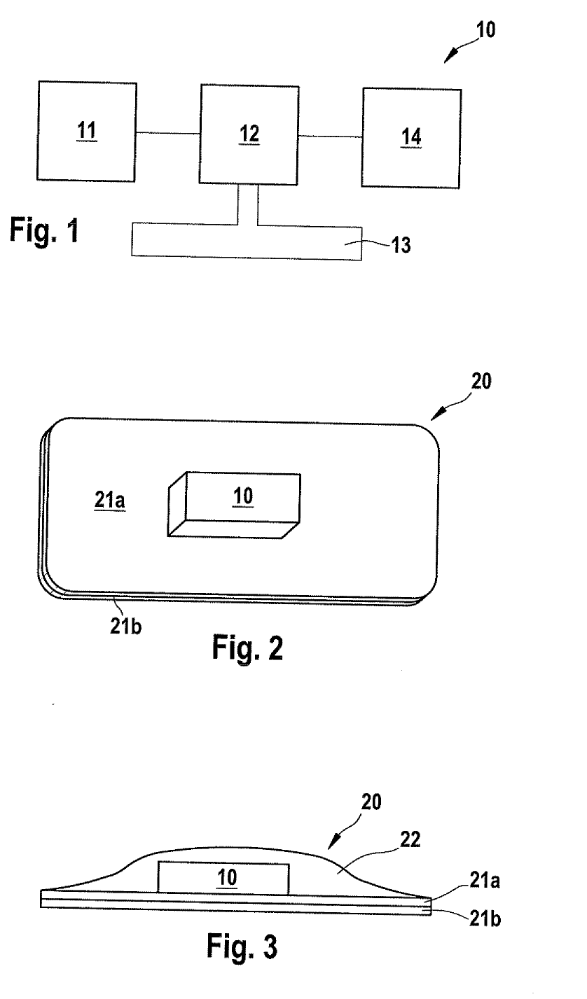

FIG. 1 shows an electronic module 10 of a patch according to the present invention. Electronic module 10 includes a motion sensor 11, an analyzer device 12, a data transmission device 13, and a power supply device 14. FIG. 1 illustrates that motion sensor 11 is electrically connected to analyzer device 12 for transmitting measured data. Furthermore, FIG. 1 shows that analyzer device 12 is electrically connected to data transmitting device 13 for transmitting, in particular exporting, analyzed data. Within the scope of the specific embodiment shown in FIG. 1, power supply device 14 is electrically connected to analyzer device 12 for the power supply, motion sensor 11 and data transmitting device 13 being indirectly supplied with power via analyzer device 12. However, it is also possible within the scope of the present invention to implement other ways and connections for the power supply. For example, it is possible to electrically connect motion sensor 11, analyzer device 12, and data transmission device 13 to power supply device 14 each via their own line for the power supply. Electronic module 10 preferably has a bendable design.

FIG. 2 shows a schematic, perspective view of a first specific embodiment of a patch according to the present invention. Within the scope of this specific embodiment, patch 20 includes an electronic module 10, a support layer 21a and an adhesive layer 21b. FIG. 2 illustrates that adhesive layer 21b is situated on one side of support layer 21a and electronic module 10 is situated on the other side of support layer 21a.

FIG. 3 shows a schematic cross section through a second specific embodiment of a patch 20 according to the present invention. Within the scope of this specific embodiment, patch 20 includes an electronic module 10, an adhesive layer 21b, a support layer 21a, and a sealing layer 22. Sealing layer 22 is preferably made of a bendable and/or elastic material. Sealing layer 22, support layer 21a, and adhesive layer 21a rest on one another in a sandwich-like manner, support layer 21a being situated between adhesive layer 21b and sealing layer 22. FIG. 3 shows that electronic module 10 is situated on support layer 21a and is covered with sealing layer 22. In this way, on the one hand, electronic module 20 may be advantageously protected from external, e.g., mechanical or chemical, effects. On the other hand, the wearing comfort is advantageously improved due to sealing layer 22, since the risk of, e.g., pieces of clothing getting caught on electronic module 10 is reduced. Within the scope of the present invention, support layer 21a may be omitted by suitably selecting the material of sealing layer 22.

What is claimed is:

1. A patch for detecting movements of a body, comprising:
a motion sensor;
an analyzer device;
a data transmission device; and
a power supply device.

2. The patch according to claim 1, wherein the patch is a disposable patch.

3. The patch according to claim 1, wherein data is readable by a stationary or mobile query device.

4. The patch according to claim 1, wherein data is readable by an RFID reader.

5. The patch according to claim 1, wherein the motion sensor is a micro-electromechanical system (MEMS).

6. The patch according to claim 1, wherein the motion sensor detects accelerations in two dimensions.

7. The patch according to claim 1, wherein the motion sensor detects accelerations in three dimensions.

8. The patch according to claim 1, wherein the analyzer device includes at least one of an integrated circuit, an application-specific integrated circuit and a microprocessor.

9. The patch according to claim 1, further comprising a data storage unit.

10. The patch according to claim 1, wherein the data transmission device includes an RFID transponder.

11. The patch according to claim 1, wherein the motion sensor, the data transmission device, the power supply device and the analyzer device have a bendable design.

12. The patch according to claim 9, wherein the data storage unit has a bendable design.

13. The patch according to claim 1, further comprising a sealing layer, which is at least one of bendable and elastic.

14. The patch according to claim 13, wherein the motion sensor, the data transmission device, the power supply device, the analyzer device and a data storage unit are situated in the sealing layer or are covered by the sealing layer.

15. The patch according to claim 1, wherein the patch is used for at least one of the following:
detecting movements of a human in at least one of sports, fitness, elderly care, and medicine,
detecting movements of an animal,
monitoring movements of sleeping, bed-ridden, dementia, or coma patients,
measuring muscular tremors, and
adjusting medication of Parkinson's patients.
